(19) United States
(12) Patent Application Publication
(10) Pub. No.: US 2016/0100912 A1
(43) Pub. Date: Apr. 14, 2016

(54) METHODS, INSERTS, AND SYSTEMS USEFUL FOR ENDODONTIC TREATMENT

(71) Applicants: Todd Geisler, Edina, MN (US); Graham Geisler, Edina, MN (US); Jamison Geisler, Edina, MN (US)

(72) Inventors: Todd Geisler, Edina, MN (US); Graham Geisler, Edina, MN (US); Jamison Geisler, Edina, MN (US)

(21) Appl. No.: 14/882,017

(22) Filed: Oct. 13, 2015

Related U.S. Application Data
(63) Continuation of application No. 13/811,567, filed on Mar. 26, 2013, now abandoned.
(60) Provisional application No. 61/367,180, filed on Jul. 23, 2010.

(51) Int. Cl. A61C 5/04; A61K 6/00; A61C 5/06

(52) U.S. Cl. CPC A61C 5/04; A61C 5/062; A61K 6/0032; A61K 6/0067

(57) ABSTRACT

Devices, methods, inserts, and systems useful in endodontic treatments, involving, for example, a shaped regenerative endodontic insert that contains platelet-fibrin matrix; certain shaped insert embodiments are formed by molding in a cavity mold, optionally as part of a mold assembly.

### PRIORITY

This application is a continuation of U.S. Ser. No. 13/811,567, filed Mar. 26, 2013 which claims the benefit from International No. PCT/US2011/044644, which was granted an International Filing Date of Jul. 20, 2011, which in turn claims priority under 35 U.S.C. §119(e) from United States Provisional patent application having Ser. No. 61/367,180, filed on Jul. 23, 2010, by Geisler et al. and titled METHODS, INSERTS, AND SYSTEMS USEFUL FOR ENDODONTIC TREATMENT, which applications are incorporated herein by reference in their entireties.

### BACKGROUND

Endodontic procedures known as “root canal therapy” involve the pulp of a tooth contained in the hollow “root canal” of the tooth, which is the interior space contained along the length of a root of a tooth. Root canal therapy is a well-known dental procedure that involves removing a top portion of a diseased tooth, cleaning the root canal portion of the tooth and the pulp portion, filling the extripated root canal with a replacement such as a rubbery compound (e.g., gutta percha), and cementing a crown or closure to the tooth. The replacement material can be in the form of a “cone,” a “post,” or an “insert.”

The root canal portion of a tooth is present at each root of tooth. The root canal includes a hollow pulp chamber, which is the anatomical hollow space within a root of a tooth naturally inhabited by nerve tissue and blood vessels. The endodontic procedure commonly referred to as a “root canal” (i.e., “root canal therapy”) generally includes removal of the pulp structure (including nerves and blood vessels) followed by cleaning, shaping, and decontaminating the hollow space of the root canal, followed by filling the space (“obturation”) with an inert filling such as gutta percha. After the surgery the tooth is “non-vital.” A root canal procedure may be necessary to treat a tooth that has been affected by trauma (e.g., mechanical injury), infection, potential infection, or other conditions.

Researchers are now experimenting with root canal therapy that does not involve filling the hollow root canal space with inert material, but that instead causes revascularization of the pulp. These treatments can be advantageous because the treated tooth retains vitality.

### SUMMARY

The following description relates to methods, compositions, systems, articles, and kits useful for restoring a diseased or damaged tooth such that infection is inhibited or eliminated and pulp regeneration is facilitated. Specific embodiments involve root canal therapies that regenerate natural tissue in a root canal in a manner that avoids past root canal therapies that place non-vital materials within an obturated root canal, resulting in a non-vital tooth. Instead, described methods place a shaped regenerative endodontic insert into a root canal during an endodontic procedure; according to preferred such methods, the shaped insert provides the basis for tissue regeneration within the root canal subsequent to the endodontic treatment, such as regeneration of nerve, pulp, or nerve and pulp tissue within the root canal.

The ability to predictably regenerate natural tissues and create new tissues has been described as requiring three essential components: progenitor (stem) cells, morphogenetic signals (including growth factors), and a three-dimensional scaffold. These components are contained in human blood, which can optionally and preferably be provided from a patient at a time of treatment. The blood can be formed into a platelet-rich material that includes: a cell source, a three-dimensional physical scaffold in the form of protein, and morphogenetic signals present in the form of growth factors and other components capable of stimulating cellular proliferation and directing cellular differentiation. According to the invention, a shaped regenerative endodontic insert can be prepared from a platelet-fibrin matrix, e.g., derived from blood that has been concentrated into a plasma concentrate. The insert can preferably be autologous to the patient, meaning that the platelet-fibrin matrix is derived from blood of the patient being treated with root canal therapy.

The description also includes various compositions useful as regenerative endodontic inserts that include a physiologically acceptable matrix such as a platelet-fibrin matrix, optionally seeded with pulp cells and one or more optional additive selected from growth factors, vitamins, stem cells, antibiotics. The platelet-fibrin matrix is derived from a plasma concentrate, which is derived from blood, e.g., whole blood, such as a sample of the patient's own blood (optionally drawn for the purpose of producing the insert), and can be formed by a procedure that includes drawing blood, concentrating the blood into a plasma concentrate that contains fibrinogen and platelets, causing the fibrinogen to polymerize (or clot or coagulate) to produce a coagulated fibrin that with platelets of the plasma concentrate makes up a scaffold in the form of a platelet-fibrin matrix. The platelet-fibrin matrix is separated from plasma serum of the plasma concentrate and is shaped into an insert having a size and shape to allow insertion into a space of a root canal.

The total mass of the insert can depend on the amount of blood used to prepare the plasma concentrate and the efficiency of converting soluble fibrinogen within the blood sample into polymerized fibrin and a platelet-fibrin matrix. In an efficient method, a substantial portion (e.g., at least 50 percent, at least 80 percent, or at least 90 percent) of fibrinogen from a patient's drawn blood sample can be converted to coagulated fibrin or a platelet-fibrin matrix that is then shaped into the shaped regenerative endodontic insert.

The insert can be made using any useful equipment such as a mold of a size to produce an insert as described. Certain types of molds and mold apparatus can be preferred, including a mold or mold apparatus that includes a reservoir portion, an insert cavity portion, and a separator. The insert cavity portion can be a size and shape designed to mimic a size and shape of a root canal. The separator allows separation of a platelet-fibrin matrix from plasma liquid and formation of the platelet-fibrin matrix into a shaped regenerative endodontic insert by collecting the platelet-fibrin matrix in the insert cavity portion.

A mold or mold apparatus can additionally include a second separator that can further mechanically compress, squeeze, or adjust the size or shape of the insert after the insert has been concentrated in the insert cavity portion. The mold or mold apparatus can optionally include a holder that is capable of mechanically grasping and holding the insert for removal from the mold and manipulation during an endodontic procedure. The holder can optionally be a feature of a holder tool that additionally includes a handle, and that can be removed from the mold or mold apparatus for convenient and sterile manipulation of the insert, for placement of the insert within a root canal of a patient under endodontic treatment.

Also described herein are evacuated test tubes that contain one or more additive or additives that may optionally be combined with a blood sample or a plasma concentrate, to produce a regenerative endodontic insert as described. According to preferred methods these additives can be included in an evacuated test tube upon drawing blood from a patient into the evacuated test tube. The drawn blood, containing the additives pre-placed in the evacuated test tube, can then be used to produce a plasma concentrate, which can be used to produce a regenerative endodontic insert as described.

In one aspect the invention relates to a shaped regenerative endodontic insert that includes a platelet-fibrin matrix containing platelets and coagulated fibrin. The shaped insert is shaped to fit a root canal.

In another aspect the invention relates to a regenerative endodontic insert that includes a platelet-fibrin matrix containing platelets, coagulated fibrin, and additive selected from the group consisting of: β-glycerophosphate, vitamin D₃, dexamethasone, and combinations thereof.

In another aspect the invention relates to a method of preparing a shaped regenerative endodontic insert. The method includes: providing blood; concentrating the blood to provide a plasma concentrate that contains plasma liquid and platelet-fibrin matrix, the platelet-fibrin matrix including platelets and coagulated fibrin; separating plasma liquid from the platelet-fibrin matrix; and forming the platelet-fibrin matrix into a shaped regenerative endodontic insert.

In another aspect the invention relates to a method of performing an endodontic procedure. The method includes: providing a shaped regenerative endodontic insert as described, and placing the insert in a root canal.

In another aspect the invention relates to a method of preparing a shaped endodontic insert. The method includes: providing a mold assembly that includes a mold having a reservoir portion and an insert cavity portion; locating plasma concentrate in the reservoir portion, the plasma concentrate containing plasma liquid and platelet-fibrin matrix; separating plasma liquid from the platelet-fibrin matrix, within the reservoir portion.

In yet another aspect the invention relates to an evacuated test tube containing growth factor. The test tube can contain, for example, a combination of growth factor and vitamin, such as a combination of β-glycerophosphate, vitamin D₃, and dexamethasone.

In yet another aspect the invention relates to a mold assembly capable of forming platelet-fibrin matrix into a shaped regenerative endodontic insert. The assembly includes a reservoir portion, an insert cavity portion, and a plunger.

### BRIEF DESCRIPTION OF THE DRAWINGS

FIG. 1A shows an embodiments of shaped endodontic insert.

FIG. 1B shows an embodiment of a shaped endodontic insert.

FIG. 1C shows an embodiment of shaped endodontic implants and a holder.

FIG. 2A shows an embodiment of a mold.

FIG. 2B shows an embodiments of a mold.

FIG. 3A shows an embodiment of a holder, mold, and insert.

FIG. 3B shows an embodiment of a holder, mold, and insert.

FIG. 3C shows an embodiment of a holder, mold, and insert.

FIG. 3D shows an embodiment of a holder, mold, and insert.

FIG. 4A shows an embodiments of a holder, a mold, and insert.

FIG. 4B shows an embodiment of a holder, mold, and insert.

FIG. 4C shows an embodiment of a holder, mold, and insert

FIG. 5A shows an embodiments of a mold and mold assembly.

FIG. 5B shows an embodiment of a mold and mold assembly.

FIG. 6 shows an embodiment of a mold and mold assembly.

### DETAILED DESCRIPTION

The following description identifies methods, compositions, articles, devices, kits, combinations, and apparatus, useful in endodontic methods of restoring a diseased or damaged tooth such that infection is inhibited or eliminated and tissue (e.g., pulp tissue, nerve tissue, or pulp and nerve tissue) regeneration is facilitated. Specifically described embodiments of the invention relate to root canal therapies that include placement of a shaped regenerative endodontic insert within a root canal of a patient. The shaped regenerative endodontic insert is prepared to include a platelet-fibrin matrix processed to be shaped into a size and form that fits the interior space of a patient's root canal. The invention also relates to kits, containers (e.g., evacuated test tubes), molds, and mold apparatus, separately or in combination, useful for preparing and shaping the shaped regenerative endodontic insert.

According to the invention, a shaped regenerative endodontic insert is prepared from a platelet-fibrin matrix, which can preferably be autologous to the patient, meaning that the platelet-fibrin matrix is derived from blood of the patient being treated with root canal therapy. The platelet-fibrin matrix is derived from a plasma concentrate, which is derived from blood, e.g., whole blood, preferably the patient's own blood.

Blood contains red blood cells, white blood cells, plasma (which includes water), and platelets. Red blood cells, or “erythrocytes,” normally make up 40 to 50 percent of blood volume. These cells transport oxygen from the lungs to all of the living tissues of the body and carry away carbon dioxide. The total water in blood (e.g., whole blood) is typically in the range of about 70 percent water. White blood cells (WBCs), or leukocytes are cells of the immune system involved in defending the body against both infectious disease and foreign materials. White blood cells exist in variable numbers and types but make up a very small part of blood volume, normally only about 1% in healthy people.

Plasma (or “blood plasma” or “plasma liquid”), the fourth major component of blood, is the clear to yellowish colored liquid component of blood, in which the red and white blood cells, and platelets, are suspended. Plasma is made of mostly water (e.g., at least 90 percent, 92 percent, or up to 95 percent by weigh), with minor amounts of dissolved sugar, fat, protein, hormones, nutrients, enzymes, and salt. A normal amount of plasma in blood is about 55 percent by volume. Platelets, or thrombocytes are small, irregularly-shaped anuclear cell fragments (i.e. cells that do not have a nucleus containing DNA) derived from fragmentation of precursor megakaryocytes. Platelets play a fundamental role in hemostasis, and also contain angiogenic growth factors, sometimes helpful in wound healing. Platelets are involved in hemostasis, leading to the formation of blood clots. Platelets typically make up 5 to 7 percent of blood by total volume.

Whole blood also contains proteins including fibrinogen, which is the soluble protein (a glycoprotein dissolved in the plasma) that is converted into the insoluble protein “fibrin” during formation of a blood clot. Without being bound by theory, the blood clotting process, starting from the soluble plasma glycoprotein fibrinogen, has been described as involving conversion of the fibrinogen by thrombin into the insoluble protein fibrin. Processes in the coagulation cascade are said to include activation of the zymogen prothrombin, to the serine protease thrombin, which is responsible for converting fibrinogen into fibrin. Fibrin is then cross linked by factor XIII (a transglutaminase enzyme, also referred to as “fibrin stabilizing factor”) to form a blood clot. As a normal biological function, the soluble fibrinogen can be converted into the insoluble fibrillar fibrin protein, which can form a protein “mesh” or scaffold that forms a hemostatic plug or clot (in conjunction with platelets) over a wound site. Plasma from whole blood can typically contain from 0.2 to 0.4 percent fibrinogen.

A “plasma concentrate” is a derivative of blood (e.g., whole blood) prepared by removing red and white blood cells to concentrate platelets and other non-blood cell components of the blood, including fibrinogen (optionally as coagulated fibrin) in the blood plasma (sometimes referred to as the “plasma liquid”). Typically, a step of preparing a plasma concentrate from whole blood can be carried out by centrifugation of the whole blood. A useful plasma concentrate may be concentrated from whole blood containing approximately 70 percent water, to a concentrated derivative that contains greater than 90 percent water, e.g., from 92 to 95 percent water.

A variety of different derivatives of whole blood, referred to as “plasma concentrates,” are known and are sometimes referred to with various names or designations including “platelet-rich plasma” (“PRP”), platelet-rich concentrates (“PRCs”), “platelet-leukocite gel,” “platelet-leukocite-rich plasma,” “Preparation Rich in Growth Factors” or “PRGF,” “Platelet-Rich-Fibrin” or “PRF,” and more generally and simply as “platelet-rich products.” These variations of plasma concentrates may differ based on the process used to prepare the plasma concentrate, the composition of the plasma concentrate, and possibly one or a combination of multiple components of whole blood considered a target of the preparation (e.g., growth factors, platelets, stem cells, etc.). Any of these particular types of plasma concentrates, as well as various other types of plasma concentrates, can be useful according to the methods described herein for preparing and using a shaped regenerative endodontic insert as described.

According to useful method steps, a plasma concentrate can be prepared by taking a blood sample, such as by drawing blood from a patient to be treated by an endodontic procedure. The sample may be acquired by standard blood drawing methods, e.g., venipuncture, such as by use of a needle and syringe, test tube, or evacuated test tube (e.g., a Vacutainer® brand test tube container marketed by Becton and Dickinson). An evacuated test tube can have a volume of 5 to 20 milliliters, such as from 8 to 15 milliliters (a typical volume can be about 10 milliliters), and can be in the form of an evacuated glass cylinder sealed at an open end by a plastic or rubber stopper or membrane that can be pierced by a needle for a venipuncture procedure. “Evacuated” can mean an evacuated test tube that has been depressurized to exhibit a reduced pressure, meaning a pressure that is less than 1 atmosphere, such as less than 0.5 atmosphere, or less than 0.2 or 0.1 atmosphere. Blood may be drawn from a patient to provide an amount (volume) of blood that is useful to produce one or more regenerative endodontic insert or inserts. For example, to produce a single regenerative endodontic insert, a required or useful volume of blood may be in the range from about 5 to 15 milliliters, e.g., from 8 to 12 milliliters. To produce multiple (e.g., up to four) regenerative endodontic inserts as may be required in certain endodontic procedures, a required or useful volume of blood may be in the range from up to about 40 milliliters, approximately 8 to 12 milliliters per insert.

A plasma concentrate can typically be formed by centrifugation of whole blood, but other methods of separating blood cells from plasma liquid could also be used to produce a plasma concentrate. By centrifugation, a variety of centrifuge systems can be useful, including benchtop or laboratory-scale centrifuges useful to centrifuge a blood sample contained in a test tube, such as a test tube having a volume of from 5 to 15 milliliters. A variety of commercially available benchtop or laboratory scale centrifuges are known and can be useful for performing methods as described.

Centrifugation conditions including timing, speed, and total gravitation to which a blood sample is exposed during centrifugation, can be any conditions useful to produce a desired plasma concentrate. Exemplary conditions useful to produce a plasma concentrate from whole blood by centrifugation of approximately 10 milliliters of whole blood, in a test tube of approximately 10 milliliter volume, can be a speed of 1500 to 2000 revolutions per minute (rpm) (e.g., from 1700 to 1900 rpm), for a time in the range from 8 to 12 minutes, e.g., from 9 to 11 minute.

According to various embodiments of the described methods, a blood sample can be centrifuged in the presence of one or more added material, ingredient, or additive that may be added to a blood sample during blood draw, or to a plasma concentrate. Addition of the added ingredient to the blood sample or plasma concentrate may be accomplished by any method, such as by including the ingredient in an evacuated test tube into which the blood is drawn. The added ingredient may be any material useful for processing the blood sample into a plasma concentrate, or processing a plasma concentrate into a shaped regenerative endodontic insert. Examples of added ingredients that may be particularly useful include one or more antibiotic; one or more vitamin such as vitamin D₃ (“cholecalciferol” or “calciol”); one or more growth factor such as β-glycerophosphate (β-Gly), and dexamethasone or “Dex” (e.g., L-dexamethasone or “L-Dex”), or a combination of two or more of these.

Vitamin D₃ is a potent calcitropic hormone that regulates calcified tissue metabolism, and also upregulates certain extracellular matrix proteins. Vitamin D₃ has different forms, with the active form (and the one preferred for use in the compositions and methods described herein) being sometimes referred to as Calcitrol or 1,25-dihydroxyvitamin D₃. Vitamin D₃ (e.g., the active form, 1,25-dihydroxyvitamin D₃) can be added to a blood sample or a plasma concentrate in any useful or desired amount.

β-glycerophosphate (β-Gly), β-Gly is a chemical that promotes dentin sialoprotein (DSP) expression in explant cultures of human dental pulp cells and serves as a local source of inorganic phosphate ions. β-Gly can be added to a blood sample or a plasma concentrate in any useful or desired amount.

Dex (dexamethasone, or “Dex”) and L-Dex are corticosteroids that upregulate DSP-phosphophorin expression. Dex or L-Dex can be added to a blood sample or a plasma concentrate in any useful or desired amount.

Before centrifugation, an anticoagulant such as sodium citrate (among others) may optionally be added to a blood sample, but alternate methods as described can exclude addition of an anticoagulant to a blood sample before centrifugation to form a plasma concentrate. In the absence of anticoagulant, a blood sample (e.g., whole blood) that is centrifuged to form a plasma concentrate will experience coagulation of fibrin during the centrifugation process. Consequently, the plasma concentrate produced by centrifugation to remove white and red blood cells will produce a plasma concentrate that includes plasma liquid that contains coagulated fibrin (and platelets). If anticoagulant is added to a blood sample prior to preparing a plasma concentrate, a coagulant (e.g., calcium chloride, among others) can be added at a later time, as desired, to cause coagulation of the fibrinogen into coagulated fibrin.

Stem cells or progenitor cells can also be added to a blood sample or to a plasma concentrate, but may also be excluded from a blood sample or a plasma concentrate as described.

An example of a useful evacuated test tube can include a combination of Vitamin D₃, β-glycerophosphate (β-Gly), and Dex (e.g., L-Dex).

Fibrinogen in a plasma concentrate may be either be in a form of coagulated fibrin (if prepared in the absence of an anti-coagulant) or may be non-coagulated (if prepared in the presence of an anti-coagulant), as desired. According to alternate versions of the described methods, anticoagulant may be added to blood prior to centrifugation of blood to form a plasma concentrate, to prevent coagulation during centrifugation. Or, fibrin may be allowed to coagulate during centrifugation of blood in forming a plasma concentrate. Upon conversion of fibrinogen to coagulated fibrin, the coagulated fibrin functions as a mesh or scaffold of insoluble, polymerized fibrin molecules that acts to contain (e.g., “enmesh”) platelets within the mesh or scaffold structure; in this form, the collective composition of coagulated fibrin and enmeshed platelets is referred to as a “platelet-fibrin matrix.”

A “platelet-fibrin matrix” refers to a composition derived from blood by coagulating fibrinogen to produce fibrin (“coagulated fibrin”), in the presence of platelets, to produce a three-dimensional fibrin mesh or fibrin network that enmeshes the platelets. The platelet-fibrin matrix may be prepared from a blood sample during preparation of a plasma concentrate from the blood sample, or following preparation of a plasma concentrate. A platelet-fibrin matrix typically forms within a plasma concentrate in the form of a dispersed three-dimensional matrix or “cloud” of dispersed fibrin. The fibrin (coagulated fibrin) is a matrix or mesh that includes interstices that enmesh platelets. When initially generated in a plasma concentrate, the platelet-fibrin matrix forms a cohesive cloud or web within the plasma liquid.

Examples of platelet-fibrin matrix compositions include those sometimes referred to as human autologous fibrin matrices (hAFM). hAFM compositions are understood to contain platelets, coagulated (polymerized) fibrin, plasma fluid, and autogenous growth factors. According to preferred methods described herein, a patient can provide blood from which a platelet-fibrin matrix can be prepared, such as a human autologous fibrin matrix, and the platelet-fibrin matrix can be used to prepare a shaped regenerative endodontic insert that is placed in a root canal of the patient in a root canal therapy.

A method as described includes forming a platelet-fibrin matrix from blood, e.g., during formation of a plasma concentrate or as a derivative of a plasma concentrate. A platelet-fibrin matrix can be formed by centrifuging blood in a manner to produce a plasma concentrate, wherein the processing causes soluble fibrinogen in the plasma concentrate to polymerize or coagulate, e.g., during centrifugation, to coagulated fibrin. Alternately, a platelet-fibrin matrix can be formed by centrifuging blood in a manner to produce a plasma concentrate in the presence of an anticoagulant, wherein processing using the anticoagulant prevents fibrinogen in the plasma concentrate to polymerize or coagulate during centrifugation; as a later step, coagulant can be added to the plasma concentrate to cause the fibrinogen to coagulate to form coagulated fibrin.

Once a platelet-fibrin matrix is formed (generally from a plasma concentrate that also contains a substantial portion of plasma liquid), a portion of the remaining plasma liquid is separated from the plasma concentrate and the platelet-fibrin matrix. According to particular examples of such methods, plasma liquid can be separated from the platelet-fibrin matrix, and the platelet-fibrin matrix can be condensed and reduced in size to form a shaped regenerative endodontic insert (e.g. an endodontic “cone”). A mold can be used to shape the regenerative endodontic insert in a non-random fashion, and in an elongate, optionally tapered form to approximate a size and shape of a root canal of a patient being treated.

Endodontic inserts and useful and preferred size and shape features are known in the endodontic arts. Examples of inserts and methods of their use are described, for example, in U.S. Pat. Nos. 5,275,562; 5,769,638; 6,262,471; 7,086,864; and 7,097,455; the entirety of each of these patent documents being incorporated herein by reference.

A shape of a regenerative endodontic insert can be as desired, such as in the form of a tapered and elongate, e.g., conical, shape having a wide end and a narrow end, with a solid, optionally tapered, cylindrical or conical elongate shaft or post extending from the narrow to the wide end. Generally, the shape of the insert can be designed to fit within an extripated root canal, by inserting the insert into the root canal from an opening in the patient's tooth, narrow end first. The insert can be sized generally for a generic root canal, or can alternately be sized based on a measured or estimated size of a root canal of a patient being treated. Standardized cones are sized to correspond to dimensions of standardized endodontic instruments. They are easily inserted into a root canal of a patient.

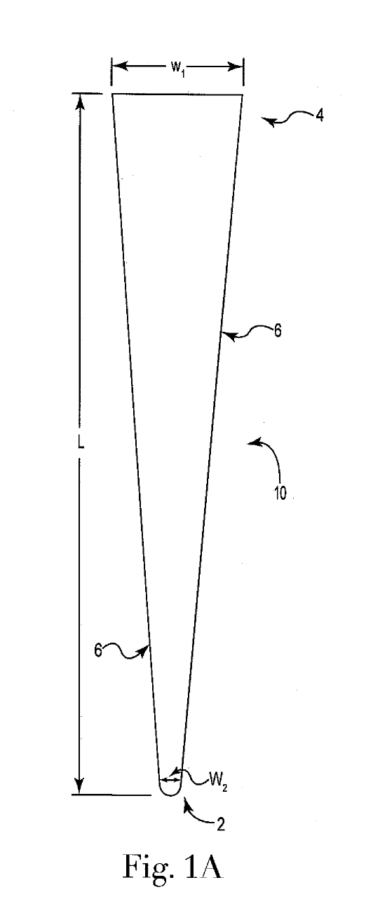

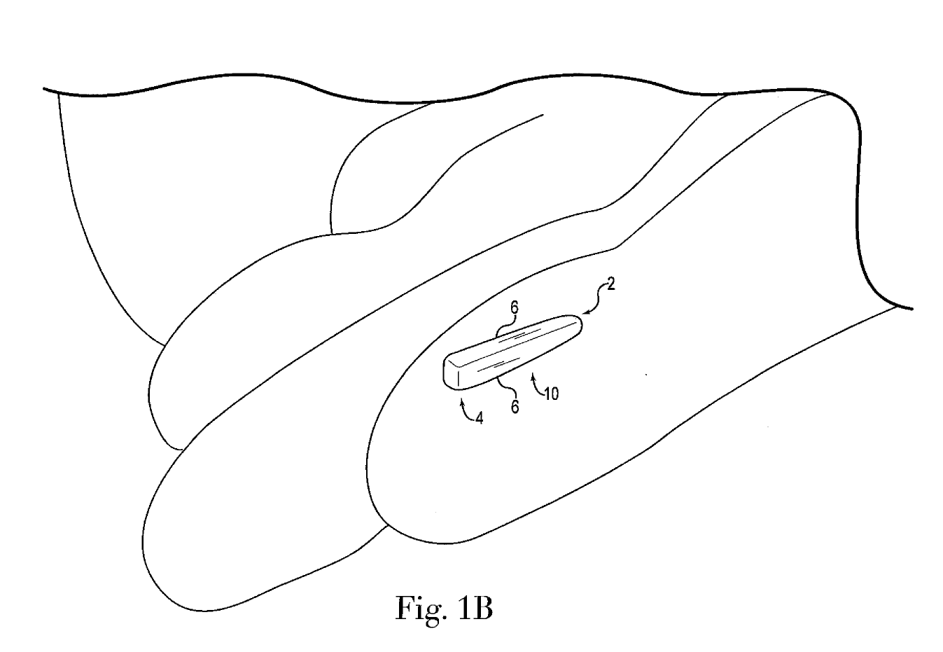

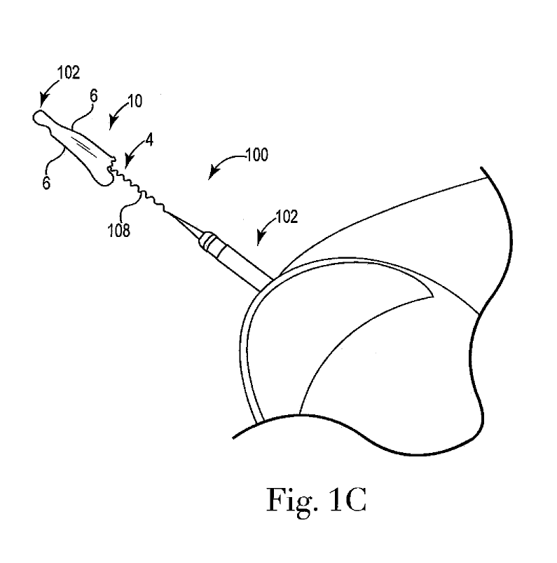

FIG. 1A show an example of a tapered (conical) insert (10) as described. An exemplary width (W₂) at or near the narrow end (2) can be in a range from 0.3 to 2 millimeters. An exemplary width (W₁) at or near the wide end (4) in a range from 1.6 to 2.4 millimeters. A total length (L) of can be as needed, with exemplary lengths being in a range from about 20 to 30 millimeters. Insert 10 is shown as including a length that includes tapered sides (6) tapered along the entire length of the insert; optionally one or more section, segment, or portion along a length of the insert may be shaped in a non-tapered manner, such as to include portion of the length that is in the form of a straight (e.g., cylindrical) section. For a method of treating a patient using an insert as illustrated, one or more insert can be prepared and used for the treatment, one insert being placed in a single root canal of a tooth. As will be understood, irregularities in the forming process due, for example, squeezing the mass of a three-dimensional network of platelet-fibrin matrix into a relatively more dense, shaped (e.g., conical) form, will not produce a shape that perfectly matches the shape of the mold into which the mass is inserted, but that will preferably approximate the shape of the mold. A mold in the form of a perfect cone can preferably produce a molded regenerative endodontic insert that approximates the cone shape of the mold, such as by having an elongate form with a relatively narrow end and a relatively wide end, but may not have a smoothly formed shaft or uniform taper to the cone body and sides, as will be understood. FIG. 1B shows an example of insert 10 having a form of a non-perfect cone, including narrow end 2, wide end 4, and sides 6 having a non-uniform taper. FIG. 1C shows an example of insert 10 having a form of a non-perfect cone, including narrow end 2, wide end 4, and sides 6 having a non-uniform taper, with insert 10 being held on corkscrew-style holder 108 of holder tool 100, tool 100 also including handle 102.

A shaped regenerative endodontic insert that includes a platelet-fibrin matrix can be prepared from a plasma concentrate by any method. According to one useful method, a shaped regenerative endodontic insert can be made from a plasma concentrate (including a platelet-fibrin matrix having coagulated fibrin) by use of a mold that shapes the platelet-fibrin matrix into an endodontic insert, while also separating the platelet-fibrin matrix from plasma liquid.

Generally, a method of molding a shaped endodontic insert can be performed by placing a plasma concentrate into a mold or that includes a cavity having a shape that matches a desired size and shape of a the desired endodontic insert. A plasma concentrate is provided, as described, containing coagulated fibrin and platelets (collectively a “platelet-fibrin matrix”), in the form of a network suspended in plasma liquid. Also provided is a mold having a cavity with a size and shape of a desired insert (an “insert cavity”). The mold can also include an additional portion, e.g., a “reservoir” or “reservoir portion” to contain plasma liquid as the platelet-fibrin matrix is separated from the plasma liquid. A mold can be used with or can include means for separating coagulated fibrin from plasma liquid, e.g., by any mechanical separation technique. As an example, a mold can include a separator in the form of a plunger (e.g., an elongate body) that can fit into a portion of the mold such as a reservoir portion, an insert cavity portion, or both. A separator (e.g., plunger) can be advanced into a portion of a mold such as a reservoir portion or an insert cavity portion, and can allow passage of plasma liquid but not passage of coagulated fibrin. For example a separator can include an opening, aperture, passage, sieve, screen, or other structure or structures permeable to liquid but not to coagulated fibrin. Accordingly, a separator can be advanced through a portion of a mold to direct coagulated fibrin into a smaller portion (e.g., a sub-portion) of the mold while allowing plasma liquid to remain in a section of mold from which coagulated fibrin is removed, thereby separating the coagulated fibrin from the plasma liquid by condensing the coagulated fibrin and isolating the coagulated fibrin in a portion of the mold.

Plasma concentrate is placed into a mold. Fibrin of the plasma concentrate can be coagulated prior to placing the plasma concentrate in the mold or after placing the plasma concentrate in the mold, as desired. The plasma concentrate contains fibrin (and platelets, etc.) from a blood sample optionally and preferably from a patient into which an insert to be derived from the plasma concentrate will be placed. The amount of fibrin and platelets (platelet-fibrin matrix) will be determined by factors that include the concentration of fibrin and platelets in the plasma concentrate, and the volume of plasma concentrate placed in the mold. Substantially all (e.g., greater than 90 percent) of the coagulated fibrin can preferably be separated from the plasma liquid to form the endodontic insert. As exemplary amounts of materials useful for producing an endodontic insert as described, 8 to 10 milliliters of whole blood may produce 4 to 5 milliliters of plasma concentrate; this amount of plasma concentrate can include a sufficient amount of fibrinogen to produce an insert as described herein (e.g., having dimensions as shown at FIGS. 1A, 1B, and 1C, and described at related text).

Separation and shaping of the platelet-fibrin matrix can be performed by any separation and molding techniques, including substantially mechanical methods.

Within the plasma concentrate, the platelet-fibrin matrix is in the form of a solid (fibrous) web or cloud suspended and dispersed in a three-dimensional web, having interstitial spaces, throughout the plasma liquid. This mass of platelet-fibrin matrix can be mechanically reduced and compressed in size by squeezing or condensing the solid mass into a more dense fibrous mass within the plasma liquid. Compressing the mass of platelet-fibrin matrix also separates the platelet-fibrin matrix from plasma liquid; compressing the mass of platelet-fibrin matrix into a space (e.g., mold) that is shaped and sized in the form of a regenerative endodontic insert as described, will cause the platelet-fibrin matrix to take on the approximate form of the space into which the matrix is squeezed, thereby producing a shaped regenerative endodontic insert.

Certain new molding apparatus and tools can be particularly useful for performing steps of molding a platelet-fibrin matrix into a regenerative endodontic insert shaped to fit into a space of a root canal. While the present description contemplates that other methods and tools will also be available and useful to carry out the described methods, following are examples of useful tools and methods of their use, in forming shaped inserts as described.

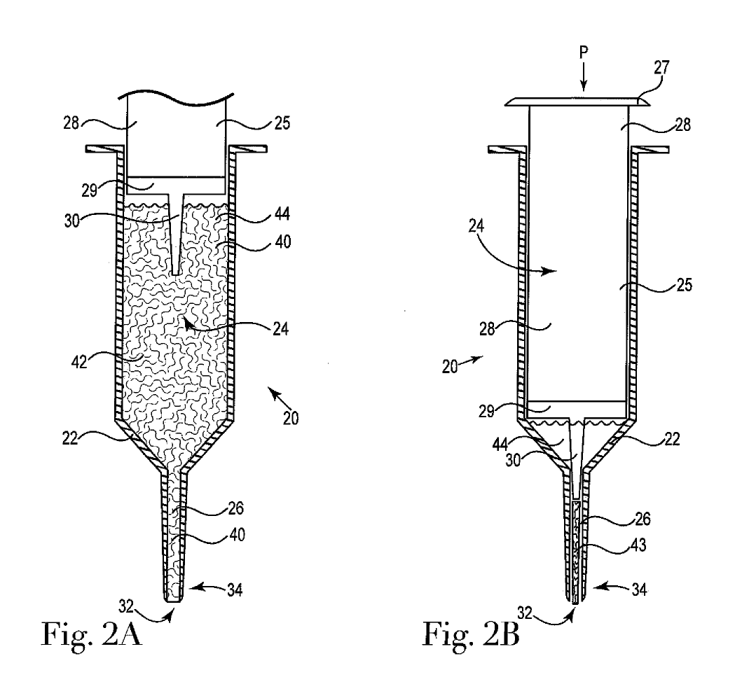

An example of a useful mold is shown at FIG. 2A and 2B. FIG. 2A shows a mold in the form of a syringe or syringe-type mold 20 that includes syringe body 22 and plunger 25. Syringe body includes reservoir portion 24 (illustrated to be an “upper” cylindrical portion of body 22) and insert cavity portion 26. Reservoir portion 24 is in fluid communication with insert cavity portion 26, which includes opening 32 at end 34. Plunger 25 includes shaft 28, plunger end 29, and extension 30; extension 30 is sized to fit within a portion of insert cavity portion 26.

In use, plasma concentrate 40 is placed into mold (syringe) 20 and fills reservoir portion 24 and insert cavity portion 26. See FIG. 2A. (Opening 32 may be capped or otherwise closed, or left open.) Platelet-fibrin matrix 42 (shown as lines in plasma concentrate 40), as illustrated at FIG. 2A, including platelets and coagulated fibrin, is suspended as a three-dimensional matrix throughout plasma liquid 44. Coagulated fibrin 42 of the platelet-fibrin matrix may be pre-coagulated (coagulated before plasma concentrate 40 is placed into mold 20) or may be coagulated (by addition of coagulant) within mold 20 after placing plasma concentrate 40 into mold 20.

Platelet-fibrin matrix 42 separated from plasma liquid 44 using plunger 25. For example, plunger 25 may be advanced into reservoir portion 24 by placing pressure (P) on plunger end 27. See FIG. 2B. A surface of plunger end 29 places pressure upon platelet-fibrin matrix 42 and plasma liquid 44, which results in flow of platelet-fibrin matrix 42 and plasma liquid 44 into insert cavity portion 26. Plasma liquid 44 can flow through insert cavity portion 26 and exit mold 20 through opening 32. Opening 32 is of a size that allows flow of plasma fluid 44 through opening 32, but platelet-fibrin matrix 42 is held up in insert cavity portion 26, not passing through insert cavity portion 26 or out of opening 32. By using plunger 25 to push plasma concentrate 40 toward opening 32, platelet-fibrin matrix 42 becomes squeezed and compressed into the space of insert cavity portion 26, and takes on the approximate shape of insert cavity portion 26, which is a shape of a regenerative endodontic insert as described herein (e.g., at FIG. 1 and related text). The regenerative endodontic insert (43, i.e., compressed platelet-fibrin matrix 42) will be generally of the form of the insert cavity portion, but will not necessarily be identical in shape or size. Irregularities in the forming process due, for example, to compression of a not-perfectly-homogenous platelet-fibrin matrix 42 into insert cavity portion 26, will not produce a shape that perfectly matches the shape of insert cavity portion 26, but that approximates the shape of cavity 26. An insert cavity portion 26 in the form of a perfect cone will produce an insert that approximates the conical mold, such as by having an elongate form with a relatively narrow end and a relatively wide end, but may not have a smoothly formed shaft or uniform taper to the cone body.

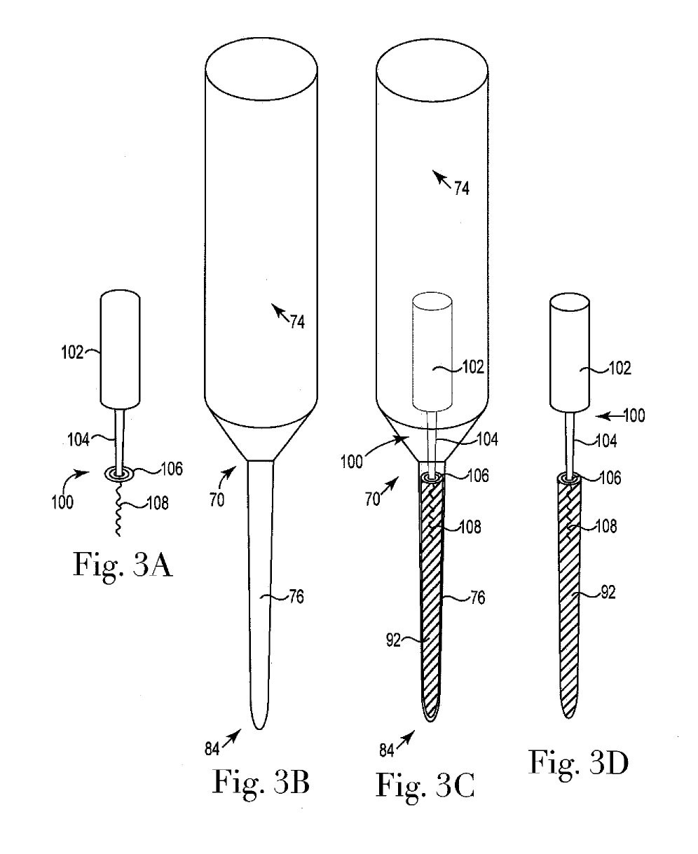

Another example of a useful mold and mold apparatus is shown at FIGS. 3A, 3B, 3D, and 3D. The illustrated mold apparatus includes a holder that is part of a holder tool. The holder securely but removably holds a shaped endodontic insert by any mechanical mode such as a spring, corkscrew, tine, hook, barb, opposable moveable or stationary jaws, or the like, any of which may optionally be stationary or moveable between open and closed positions. The holder may be fixedly or removably secured directly to a plunger end, or may optionally be part of a holder tool that can be removably attached and detached from an end of a plunger or a component of a plunger. A holder and holder tool are both optional features of a plunger, mold, mold assembly or combination or assembly of a mold or molding kit or system, and can be used for purposes of removing a shaped regenerative endodontic insert from an insert cavity portion of a mold, and also to manipulate the shaped regenerative endodontic insert during an endodontic procedure. A handle of a holder tool can removably attach to an end of a plunger. After formation of a shaped endodontic insert within a mold, the plunger can be removed with the holder or holder tool attached to the end of the plunger, and the shaped insert attached to the holder of the holder tool. A removable holder tool can be removed from (detached from) the end of the plunger, and a surgeon can manipulate the handle of the holder tool without touching the shaped insert, to manipulate the shaped insert attached to the holder of the holder tool, for example to allow sterile placement of the shaped insert into a root canal of a patient undergoing an endodontic procedure.

An optional feature of a mold apparatus as described can be a dual-action plunger. A dual-action plunger can include design features that allow formation of a shaped insert by separation of a platelet-fibrin matrix from plasma liquid, and formation and compression of a platelet-fibrin matrix into a shaped insert, in a multi-step sequence. One step of a separation and formation and compression sequence can be to separate plasma liquid from platelet-fibrin matrix in a reservoir of a mold, while pushing the platelet-fibrin matrix into an insert cavity portion of the mold, thus compressing the mass of platelet-fibrin matrix and forming the compressed platelet-fibrin matrix into what can be referred to as a shaped regenerative endodontic insert. A second step can be to subsequently adjust the size or density of the insert by further compression of the insert and separation of the insert from plasma liquid, by further pressing the insert into the insert cavity portion of the mold, as desired, for example to adjust (reduce) the size (especially the length) of the insert, necessarily also causing a change (increase) in the density (mass per size, e.g., volume) of the insert. A dual-action plunger used in conjunction with a mold as described generically or specifically herein, can be used to perform both of these steps. A dual action plunger can also optionally include a holder, the holder optionally and preferably being a component of a holder tool that can removably attach to the dual action plunger. Of course alternate methods can be performed using any equipment that is useful to perform these separating and adjusting steps in any desired manner.

Referring to figure FIGS. 3A, 3B, 3C, and 3D, mold 70 includes mold body 72 including reservoir portion 74 (illustrated to be an “upper” cylindrical portion of body 72) and insert cavity portion 76. Reservoir portion 74 is in fluid communication with insert cavity portion 76, which includes closed end 84.

Holder tool 100 includes handle 102, optional shaft 104, separator 106, and holder 108. Handle 102 can be designed to removably attach to an end of a plunger (not shown), e.g., a dual action plunger. Shaft 104 is an optional feature that can be included to create separation between handle 102 and separator 106 and holder 108. Separator 106 allows passage of plasma liquid, but not passage of coagulated fibrin.

Generally, a separator (e.g., 106) can include an opening, aperture, passage, sieve, screen, or other structure or structures permeable to liquid but not to coagulated fibrin. Optionally and as illustrated, separator 106 may be of a variable size (diameter) so that when separator 106 is advanced into insert cavity portion 76—which has a tapered, narrowing-diameter shape—the diameter of separator 106 will can change as the diameter of insert cavity portion 76 narrows (as separator 106 advances toward closed end 84). As the diameter of separator 106 is reduced, separator 106 continues to be capable of allowing plasma liquid to pass through separator 106, while a solid material such as platelet-fibrin matrix 92 does not pass, but becomes pressed farther into insert cavity portion 92. FIG. 3A illustrates holder tool 100 and features thereof. FIG. 3B illustrates mold 70 and features thereof. FIG. 3C illustrates mold 70, holder tool 100, and shaped insert 92 comprising platelet-fibrin matrix derived from plasma concentrate. FIG. 3D illustrates shaped insert 92 held by holder 108 of holder tool 100, after removing holder tool 100 from mold 70.

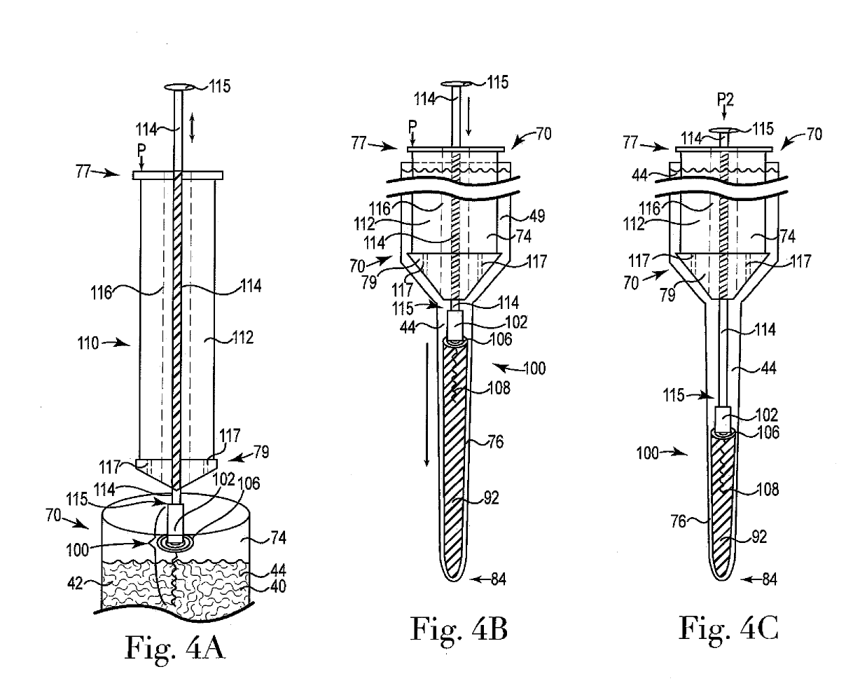

FIGS. 4A, 4B, and 4C illustrate dual-action plunger 110, including outer shaft 112 and inner shaft 114. Outer shaft 112 includes elongate cylindrical channel 116. Inner shaft 112 moveably resides within channel 116 and can be moved longitudinally (see arrows) within channel 116. Apertures or channels 117 extend longitudinally through plunger end (separator) 79 to allow plasma liquid 44 of plasma concentrate 40 to pass through plunger end (separator) 79, without allowing solid coagulated fibrin 42 to pass; apertures 117 are shown as distinct channels, but plunger end (separator) 79 may alternately include any other type of separator such as a sieve, mesh, screen, or other separator or separating means capable of separating coagulated fibrin from plasma liquid by passing the separator through concentrated plasma contained in a mold or portion of a mold. Plunger end 119 of inner shaft 114 is removably attached to holder tool 100, which includes handle 102, optional separator 106, and holder 108. Optionally and as illustrated, holder tool 100 does not require a shaft connecting holder 108 or separator 106, to handle 102.

Dual-action plunger 110 can be used with mold 70 by placing plasma concentrate 40 into mold 70 (see FIG. 4A). Plasma concentrate 40 fills reservoir portion 74 and insert cavity portion 76. Platelet-fibrin matrix 42 includes platelets and coagulated fibrin as a three-dimensionally suspended network of web suspended in plasma liquid 44. Coagulated fibrin of platelet-fibrin matrix 42 may be pre-coagulated (coagulated before plasma concentrate is placed into mold 70) or may be coagulated (by addition of coagulant) within mold 70 after placing plasma concentrate 40 into mold 70. By advancing dual-action plunger 110 into reservoir portion 74 with pressure (P) placed on plunger end 77, platelet-fibrin matrix 42 is separated from plasma liquid 44. A surface of plunger end (separator) 79 presses into platelet-fibrin matrix 42 and plasma liquid 44 of plasma concentrate 40. Plasma liquid 44 flows through apertures 117 (or like passages of a separator) and remains in reservoir portion 74 as plunger 110 advances along the length of reservoir portion 74 toward insert cavity portion 76. Platelet-fibrin matrix 42 does not pass through apertures 117 and is pushed toward and collected into insert cavity portion 76 as plunger 110 advances toward insert cavity portion 76. Plunger end 79 pushes and compresses platelet-fibrin matrix 42 into insert cavity portion 76, where platelet fibrin matrix 42 becomes squeezed and condensed into the space of insert cavity portion. 76 and takes on shape features of cavity portion 76, which is a shape of a regenerative endodontic insert as described herein (e.g., at FIGS. 1A, 113, and 1C, and related text). Regenerative endodontic insert 92 will be generally of the form of the insert cavity portion, but will not be identical in shape or size. As shown at FIG. 4B, shaped insert 92 has been formed by advancing outer shaft 112 of dual-action plunger 110 through reservoir portion 74, to push platelet fibrin-matrix 42 into insert cavity portion 76, while a large portion of plasma liquid 44 becomes separated from platelet-fibrin matrix 42 (a substantial portion of plasma liquid 44 remains within reservoir portion 74, which now contains substantially no platelet-fibrin matrix). As shown at FIGS. 4B and 4C, after the described advance of outer shaft 112, inner shaft 114 can be independently advanced farther into insert cavity 76, toward end 84, to cause separator 106 to adjust the size of shaped insert 92. At FIG. 4B, shaped insert 92 has a length nearly the length of insert cavity portion 76. By advancing separator 106 a greater distance into insert cavity portion 76 (see FIG. 4C)—performed by placing pressure (P2) on plunger end 119 of inner shaft 114—the length of shaped insert 92 is reduced, compressing the mass of platelet-fibrin matrix that forms shaped insert 92 and consequently increasing the density (mass per size) of shaped insert 92.

A subsequent step can be to remove dual-action plunger 110 from mold 70. Dual action plunger 110, including holder tool 100 with holder 108, can be removed from mold 70, with shaped insert 92 attached to holder 108. Handle 102 of holder tool 100 can then be removed from plunger end 119 of inner shaft 114, to provide holder tool 100 with shaped insert 92 attached to holder 108 (e.g., as shown generally at FIGS. 1C and 3D). Holder tool 100 can be used to manipulate shaped insert 92, such as by placing shaped insert 92 into a root canal of a patient during an endodontic treatment. Other necessary steps of the treatment can be performed subsequently, such as closure of the root canal.

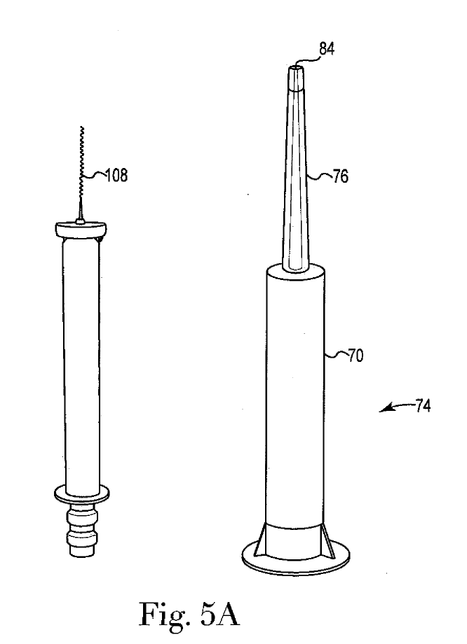

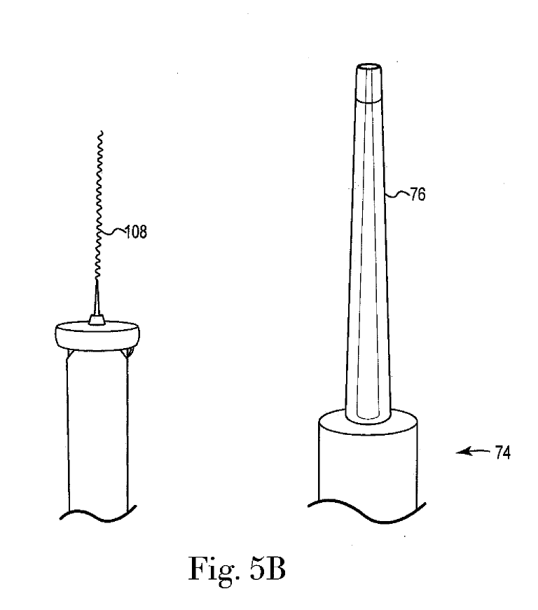

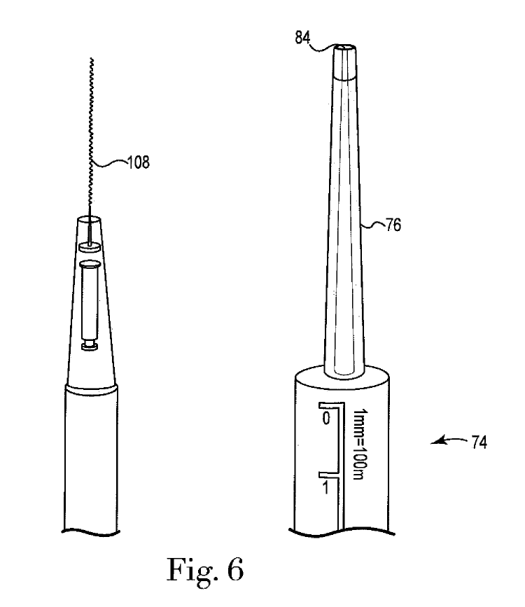

Alternate embodiments of mold and mold assemblies are shown at FIGS. 5A, 5B, and 6.

1. A compressed, shaped regenerative endodontic insert comprising a platelet-fibrin matrix comprising platelets and coagulated fibrin, wherein the compressed shaped insert is located in a mold cavity, the mold cavity also containing plasma liquid, and the compressed shaped insert has an elongate tapered shaped adapted to fit a root canal.

2. A shaped insert according to claim 1, wherein the insert is an endodontic insert and the shape comprises a tapered cone.

3. A shaped insert according to claim 1 wherein the insert has an elongate shape including a narrow end, a wide end, a length between the narrow end and the wide end, a diameter at or near the narrow end in a range from 0.3 to 2 millimeters, a diameter at or near the wide end in a range from 1.6 to 2.4 millimeters, wherein the length is from 15 to 30 millimeters and the insert includes a taper along the length.

4. A shaped insert according to claim 1 comprising additive selected from the group consisting of: growth factor, antibiotic, a vitamin, stem cells, and combinations of these.

5. A shaped insert according to claim 1 comprising the platelet-fibrin matrix and an ingredient selected from the group consisting of: β-glycerophosphate, vitamin D₃, dexamethasone, and combinations thereof.

6. A shaped insert according to claim 1, excluding coagulant and excluding anticoagulant.

7. A shaped insert according to claim 1, including coagulant and anticoagulant.

8. A regenerative endodontic insert comprising a platelet-fibrin matrix comprising platelets, coagulated fibrin, and additive selected from the group consisting of β-glycerophosphate, vitamin D₃, dexamethasone, and combinations thereof.

9. A method of preparing a shaped regenerative endodontic insert according to claim 1, the method comprising:
providing blood,
concentrating the blood to provide a plasma concentrate comprising plasma liquid and a platelet-fibrin matrix, the platelet-fibrin matrix comprising platelets and coagulated fibrin, separating plasma liquid from the platelet-fibrin matrix, and
compressing the the platelet-fibrin matrix into a compressed shaped regenerative endodontic insert shaped to fit a root canal.

10. (canceled)

11. (canceled)

12. A method according to claim 9 wherein
providing blood comprises providing blood from a patient,
concentrating comprises centrifuging the blood to produce the plasma concentrate, and
the method comprises coagulating fibrinogen to produce a platelet-fibrin matrix comprising coagulated fibrin and platelets,
separating comprises placing the platelet-fibrin matrix into a mold and separating plasma liquid from the platelet-fibrin matrix, and
the method comprises molding the platelet-fibrin matrix into a shape to fit a root canal of the patient.

13. A method of performing an endodontic procedure, the method comprising:
providing a shaped regenerative endodontic insert according to claim 1, and
placing the insert in a root canal.

14-32. (canceled)

33. A shaped insert as recited at claim 1 wherein the mold comprises a tapered cylinder having two ends and tapered cylinder walls between the two ends, one end of the mold being open and the other end being closed.

34. A shaped insert as recited at claim 1 wherein the mold comprises a tapered cylinder having two ends and tapered cylinder walls between the two ends, one end having a first opening connected to a reservoir, and the other end having a second opening adapted to allow liquid to flow through but to prevent passage of the platelet-fibrin matrix.

35. A shaped insert as recited at claim 33 wherein the mold cavity comprises anticoagulant.

36. A shaped insert as recited at claim 33 wherein the anticoagulant is sodium citrate.

37. A shaped insert as recited at claim 33 wherein the mold cavity comprises coagulant.

38. A shaped insert as recited at claim 36 wherein the coagulant is thrombin.

39. A shaped insert as recited at claim 33 wherein the mold cavity comprises anticoagulant and coagulant.

40. A shaped insert as recited at claim 39 wherein the anticoagulant is sodium citrate and the coagulant is thrombin.

41. A shaped insert as recited at claim 1 wherein the mold does not contain stem cells.

42. A shaped insert as recited at claim 1 wherein the platelet-fibrin matrix does not contain stem cells.
